(19) United States
(12) Patent Application Publication
(10) Pub. No.: US 2005/0112696 A1
(43) Pub. Date: May 26, 2005

(54) Compositions, methods and assays related to secretase cleavage specificity

(75) Inventors: Thomas C. Sudhof, Dallas, TX (US); Qiming Li, Coppell, TX (US)

Correspondence Address: VINSON & ELKINS, L.L.P., 1001 FANNIN STREET, 2300 FIRST CITY TOWER, HOUSTON, TX, 77002-6760, US

(73) Assignee: The University of Texas Southwestern Medical Center, Dallas, TX (US)

(21) Appl. No.: 10/721,297

(22) Filed: Nov. 25, 2003

(51) Int. Cl. C12N 15/85; G01N 33/53; G01N 33/567

(52) U.S. Cl. 435/7.2; 435/455

(57) ABSTRACT

Compositions and methods for determining modulators of beta-secretase activity and specificity of protease activity relative to the APP family of proteins are disclosed.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. R01-MH69585-01 awarded by the National Institute of Mental Health.

### BACKGROUND OF THE INVENTION

1. Field of the Invention

The present invention relates to methods, compositions and assays for screening and identifying modulators of Amyloid-β precursor protein cleavage by β-secretases, especially the β-secretase BACE 1. More specifically, the present invention provides methods, compositions and assays for screening and identifying therapeutic agents that might be useful in treatment or prevention of Alzheimer's disease.

2. Description of the Related Art

Alzheimer's disease is a chronic neurodegenerative disorder of the brain that may lead to dementia and death and which is commonly characterized by progressive loss of memory and cognitive impairment. Pathologically, the disease is characterized by cerebrovascular amyloid deposits and neuritic plaques (“amyloid plaques”) that contain peptide cleavage products of Amyloid-β precursor protein (“APP”). The aggregation of these APP cleavage products is directly correlated to the severity of the Alzheimer's disease pathogenesis.

APP is a ubiquitous membrane protein that is physiologically processed by site-specific proteolysis (reviewed in 4-7; FIG. 1A). First, cleavage by α- or β-secretases releases the large extracellular part of APP (called APPₛ), leaving a C-terminal fragment (“CTF”) that is composed of a small remaining extracellular stub, the transmembrane region (TMR), and the cytoplasmic tail. The CTF of APP is then cut by γ-secretase at multiple positions in the TMR (Sastre, M., et al. (2001) EMBO Rep. 2, 835-841; Yu, C., et al. (2001) J. Biol. Chem. 276, 43756-43760). γ-cleavage liberates an intracellular cytoplasmic fragment referred to as amyloid precursor protein intracellular domain (“AICD”) (Kimberly, W. T., et al. (2001) J. Biol. Chem. 276, 40288-40292; Cupers, P., et al. (2001) J. Neurochem. 78, 1168-1178) that may function as a transcriptional activator (Cao, X., and Südhof, T. C. (2001) Science 293, 115-120; Gao, Y., and Pimplikar, S. W. (2001) Proc. Natl. Acad. Sci. USA 98, 14979-14984) and have other signaling roles (Lu, D. C., et al. (2000) Nat. Med. 6, 397-404; Leissring, M. A., et al. (2002) Proc. Natl. Acad. Sci. USA 99, 4697-4702). In addition, γ-cleavage generates small peptides that are derived from the TMR and adjacent extracellular sequences. These peptides include Aβ40 and Aβ42, the major components of amyloid fibrils in Alzheimer's disease (Lu, D. C., et al. (2000) Nat. Med. 6, 397-404; Leissring, M. A., et al. (2002) Proc. Natl. Acad. Sci. USA 99, 4697-4702; reviewed in Selkoe, D. J. (1998) Trends Cell Biol. 8, 447-453; Haass, C., and De Strooper, B. (1999) Science 286, 916-9 19).

The AICD of APP binds to several cytoplasmic adaptor proteins, including Fe65 and its isoforms (Fiore, F., et al. (1995) J. Biol. Chem. 270, 30853-30856; Borg, J. P., et al. (1996) Mol. Cell. Biol. 16, 6229-6241; McLoughlin, D. M., and Miller, C. C. (1996) FEBS Lett. 397, 197-200; Duilio, A., et al. (1998) Biochem. J. 330, 513-519). Transcriptional activation by AICD, as measured using heterologous DNA-binding domains that are fused to APP, requires binding of Fe65 to the AICD (Cao, X., and Südhof, T. C. (2001) Science 293, 115-120). Fe65 contains three conserved domains that are necessary for transcriptional activation: an N-terminal WW-domain (a protein module with two conserved tryptophans) whose ligand is unknown, a central amino-terminal phosphotyrosine binding domain (“PTB”) that binds to the nuclear chromosome remodeling protein Tip60, and a second C-terminal PTB domain that mediates the interaction of Fe65 with the AICD (Cao, X., and Südhof, T. C. (2001) Science 293, 115-120). The potent transactivation of transcription by the AICD/Fe65 complex via binding to Tip60 indicates that the cytoplasmic tail of APP, liberated by γ-cleavage, translocates to the nucleus as a complex with Fe65 and binds to nuclear Tip60 to transactivate transcription. (U.S. 2003/0134323, Sudhof et al. (which is incorporated herein in its entirety by reference)). Although the endogenous DNA binding domains (beyond those associated with Tip60 (Ikura, T., et al. (2000) Cell 102, 463-473)) have not been identified, this model is supported by three findings. First, the cytoplasmic tail of APP is detectable in the nucleus of cultured cells, albeit at low levels (Kimberly, W. T., et al. (2001) J. Biol. Chem. 276, 40288-40292; Cupers, P., et al. (2001) J. Neurochem. 78, 1168-1178). Second, unmodified APP together with Fe65 is a potent transactivator of Ga14 fused to Tip60 which is inactive in transcription without Fe65 and APP (Cao, X., and Südhof, T. C. (2001) Science 293, 115-120). Third, a direct target gene (the KAI1 gene encoding a tetraspanin) for the AICD/Fe65/Tip60 complex was identified in studies on how interleukin-1β activates transcription, thereby demonstrating the in vivo relevance of the original experiments using heterologous DNA binding domains (Baek, S. H., et al. (2002) Cell 110, 55-67).

APP belongs to a gene family that includes two additional members, APLP1 and APLP2 (Wasco, W., et al. (1992) Proc. Natl. Acad. Sci. USA 89, 10758-10762; Wasco, W., et al. (1993) Nat. Genet. 5, 95-99; Sprecher, C. A., et al. (1993) Biochemistry 32, 4481-4486). APP, APLP1 and APLP2 are closely related and exhibit the same domain structure (FIG. 1A). The extracellular sequences of APP and APLP's are composed of six regions: an N-terminal signal peptide (SP), a cysteine-rich domain (CRD), an acidic sequence that chelates metal ions, a Kunitz domain (Ku) that is only present in APP and APLP2 and is alternatively spliced, a cysteine-poor but conserved sequence (CAPP-Domain for ‘Central APP domain’), and a C-terminal linker that connects the extracellular domains to the transmembrane region (TMR). The percent identity (fully conserved residues) between human APP, APLP1, and APLP2 is shown in FIG. 1 only below those domains (the CRD, CAD, and AICD) that are significantly conserved among all three proteins. The location of the secreted extracellular APP/APLP fragment (APPS/APLPS) and the C-terminal cellular fragment (CTF) generated by α/β-cleavage is indicated. FIG. 1B shows the alignment of the C-terminal sequences of APP, APLP1, and APLP2 starting with the C-terminal half of the CAD and identical residues are highlighted in the CAD, TMR and AICD domains. The alternatively spliced sequences in the linker are boxed; excision of this sequence creates a recognition site for chondroitin sulfate glycosaminoglycans which are attached to the serine residue marked with an asterisk. The Aβ sequence in APP is shown in bolded underlined text; α-, β-, γ- and ε-cleavage sites are marked by arrows. The C-terminal sequences in APP and APLP's and the internal APLP1 sequence that were used to raise anti-peptide antibodies are boxed. The position of the Fe65 binding site in the AICD is indicated. Note that although the CAD and the AICD are highly conserved, the linker between the extracellular domain and the TMR exhibits no sequence similarity between APP, APLP1, and APLP2. Studies of knockout mice have revealed that APP, APLP1 and APLP2 are functionally overlapping (Heber, S., et al. (2000) J. Neurosci. 20,7951-7963). Similar to APP, APLP's appear to be cleaved by extracellular proteases followed by proteolytic processing by γ-secretase (Naruse, S., et al. (1998) Neuron 21, 1213-1221; Scheinfeld, M. H., et al. (2002) J. Biol. Chem. 277, 44195-44201; Walsh, D. M., et al. (2003) Biochemistry 42, 6664-6673). Furthermore, Ga14-fusion proteins of APLP's also transactivate Ga14-dependent transcription in an Fe65 dependent manner (Scheinfeld, M. H., et al. (2002) J. Biol. Chem. 277, 44195-44201; Walsh, D. M., et al. (2003) Biochemistry 42, 6664-6673). It is not known, however, which extracellular proteases cleave APLPs because, although the extracellular domains and the AICDs of APP and APLP's are highly homologous, the linker sequences connecting these domains exhibit no homology (FIG. 1B). Significantly it is not known if the same extracellular proteases digest APP and APLPs. This is a particularly important question for BACE 1, the primary β-secretase (Yan, R., et al. (1999) Nature 402, 533-537; Sinha, S., et al. (1999) Nature 402, 537-540; Vassar, R., et al. (1999) Science 286, 735-741; Hussain, I., et al. (1999) Mol. Cell. Neurosci., 14, 419-427; Lin, X., et al. (2000) Proc. Natl. Acad. Sci. USA, 97, 1456-1460), because it is the major extracellular processing enzyme for APP in neurons and because it initiates production of Aβ from APP (Selkoe, D. J. (1998) Trends Cell Biol. 8, 447-453; Haass, C., and De Strooper, B. (1999) Science 286, 916-9 19; Bayer, T. A., Cappai, R., Masters, C. L., Beyreuther, K. & Multhaup, G. (1999) Mol. Psychiatry 4, 524-528). Furthermore, the relationship between Alzheimer's disease and the development of amyloid plaques suggests that a promising strategy for the treatment and possible prevention of the disease would center on inhibiting the cleavage of APP, specifically inhibiting the BACE 1 mediated cleavage of APP. Thus, there remains a need in the art for refined methods of identifying agents that specifically modulate the cleavage of APP by proteolytic enzymes.

### SUMMARY OF THE INVENTION

The present invention relates to compositions, methods and assays for screening and identifying agents that modulate the proteolytic cleavage of APP by β-secretases. Furthermore, the compositions, methods and assays of the present invention relate to screening and identifying agents that differentially modulate the proteolytic cleavage of APP as opposed to APLP1 or APLP2 by β-secretases.

### BREIF DESCRIPTION OF THE DRAWINGS

FIG. 1A depicts the common domain structure of the three proteins of the APP family (APP, APLP1, and APLP2).

FIG. 1B displays the alignment of the C-terminal sequences of APP, APLP1, and APLP2 starting with the C-terminal half of the CAD.

FIG. 2A shows the results of the transactivation of transcription mediated by Ga14-Tip60 in HEK293 cells that were co-transfected with a constant amount of Fe65 plasmid (0.5 μg) and increasing amounts of APP, APLP1, or APLP2 plasmids.

FIG. 2B shows the results of the transactivation of transcription mediated by Ga14-Tip60 in HEK293 cells that were transfected with constant amounts of APP (0.5 μg), APLP1 (0.25 μg), and APLP2 plasmids (0.5 μg) and co-transfected with increasing amounts of Fe65 plasmid.

FIG. 2C shows the effect of DAPT treatment on the transactivation of transcription mediated by Ga14-Tip60 in HEK293 cells that were co-transfected with a constant amount of Fe65 plasmid (0.5 μg) and increasing amounts of APP, APLP1, or APLP2 plasmids.

FIG. 3 shows the results of a characterization of APP, APLP1 and APLP2 antibodies.

FIG. 4 demonstrates the results of an analysis of the cleavage of APP, APLP1 and APLP2 by BACE 1.

FIG. 5 demonstrates the effect of BACE 1 on the transactivation of Ga14-Tip60 dependent transcription by APLP1.

FIG. 6A depicts diagrams of the structures of APP, the neurexin 1β/APP hybrid protein (Nrx1β/APP), and the modified neurexin 1β (Nrx1β*) containing an insertion including the 7 amino acid β-secretase binding site (sequence: EVKMDAE) from APP into neurexin 1β just N-terminal to the TMR.

FIG. 6B shows the results of a comparison of the effect of BACE 1 and DAPT on CTFs produced in HEK293 control cells or transfected cells expressing APP or the neurexin 1β/APP hybrid.

FIG. 6C shows the results of a comparison of the effect of BACE 1 and DAPT on the cleavage of neurexin 1β and neurexin 1β*.

FIG. 7 shows the results of an analysis of APP cleavage by BACE 1 and ADAM 9.

FIG. 8 shows the results of an analysis of APLP1 cleavage.

### DETAILED DESCRIPTION OF SPECIFIC EMBODIMENTS

It has been discovered that BACE 1 alters proteolytic processing of APLP's in transfected cells, and thus that APLP's are substrates for BACE 1 in vivo. BACE 1 was found to cleave APLP1 near the trans membrane region (TMR), resulting in the secretion of the extracellular sequences of APLP1. In the same assay, ADAM 9, an enzyme suspected of acting on APP as an α-secretase, specifically cleaved APP but not APLP1. The present disclosure demonstrates that BACE 1 substrate recognition is specific for APP as well as APLP1 and APLP2. Furthermore, in contrast to previous reports regarding the β-secretase cleavage site in APP, a short sequence from APP (7 residues) is sufficient to confer BACE 1 cleavage onto a normally non-cleaved protein. These results indicate that BACE 1 has a coordinate function in regulating APP and APLP processing in neurons.

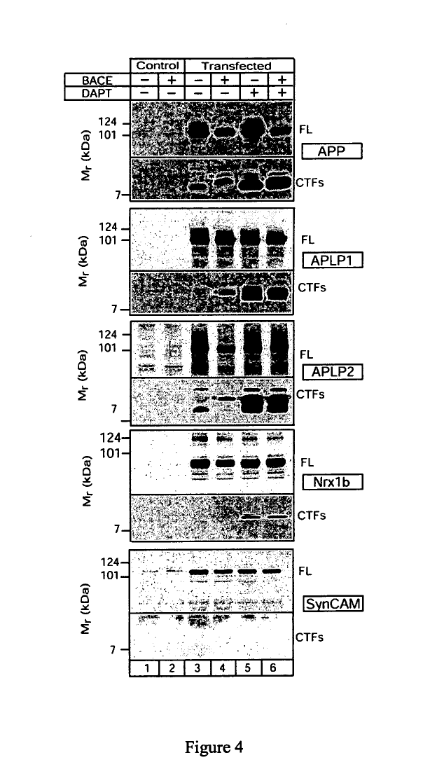

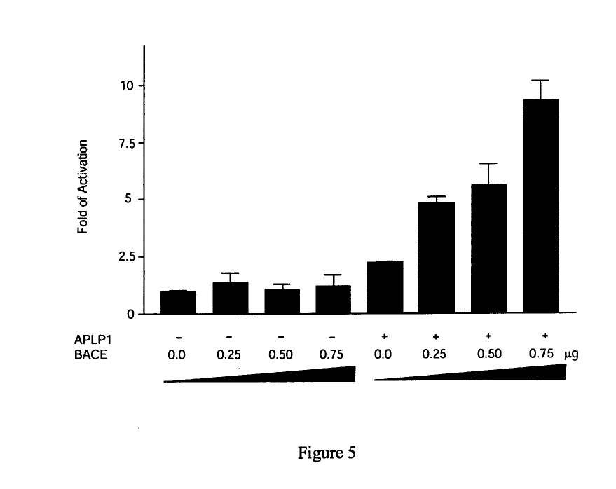

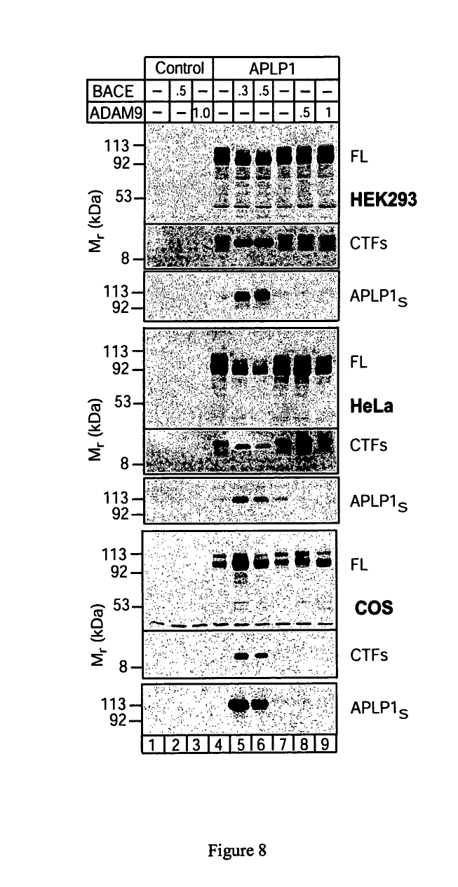

Furthermore, the present invention teaches that all three proteins are cleaved by BACE 1, the enzyme responsible for the β-secretase activity of APP cleavage and the most abundant neuronal APP cleavage protein. The evidence that BACE 1 cleaves APLP1 and APLP2 can be summarized as follows: 1. Co-transfection of BACE 1 with APLP1 or APLP2 results in the production of a new CTF (FIG. 4). 2. Increasing amounts of BACE 1 enhance transactivation of Ga14-Tip60 mediated by APLP1 (FIG. 5). 3. Co-expression of BACE 1 with APLP1 results in a large and specific increase in the secretion of APLP1 (FIG. 8) As used herein, APPₛ, APLP1ₛ and APLP2ₛ are meant to indicate a large secreted extracellular fragment of the respective proteins. 4. A newly generated antibody against a short extracellular sequence of APLP1 that is adjacent to the TMR only recognizes a secreted APLP1ₛ proteolytic fragment when BACE 1 is co-transfected with APLP1, localizing the BACE 1 cleavage site to a position next to the TMR (FIG. 8).

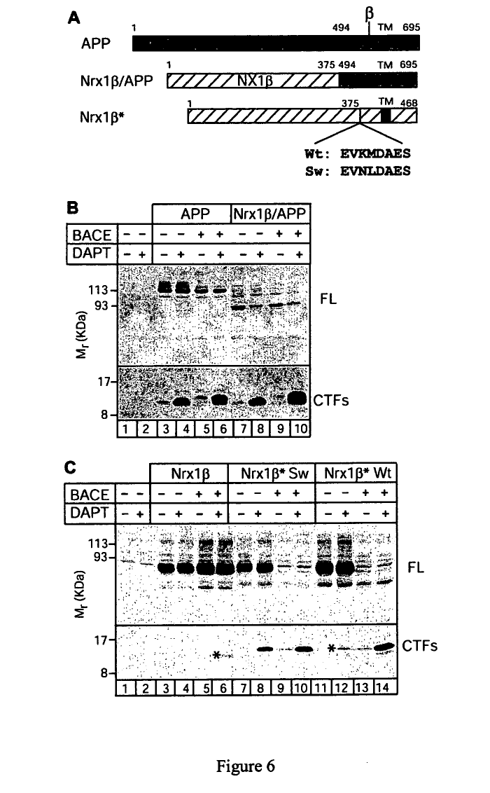

The use of a transfection assay whereby BACE 1 is co-transfected with APP or an APLP, and the effect of BACE 1 on the production of proteolytic fragments from the co-transfected protein supports this model of secretase activities. This assay was validated by demonstrating that two other neuronal cell surface proteins, neurexin 1β and SynCAM, are not cleaved by BACE 1 (FIG. 4), and by the finding that a seven residue sequence from APP, when inserted into neurexin 1β next to the TMR, is sufficient to convert neurexin 1β into a BACE 1 substrate (FIG. 6). The finding that APLP's are BACE 1 substrates similar to APP, that BACE 1 is the major extracellular neuronal secretase for APP, and that APLP's and APP are functionally redundant indicates that BACE 1 cleavage is an integral component of the functions of APP and APLP's in neurons.

Based on the linker regions of the APP and APLP proteins, BACE 1 does not appear to have a stringent substrate recognition sequence. Examination of the two β-cleavage sites in APP showed that BACE 1 prefers a hydrophobic residue preceding the cleavage site, and an acidic residue following the cleavage site. However, the β-cleavage sequences are not conserved in APLP1 or APLP2, and no similar sequence motif can be readily identified in APLP1 or APLP2. Based on this lack of sequence similarity, the cleavage of both APP and APLP's by BACE 1 is unexpected.

The demonstration that APLP's are BACE 1 substrates is consistent with the importance of BACE 1 as a processing enzyme for APP in brain and the structural and functional similarity of APP with APLP's. Since APLP's are also BACE 1 substrates, Aβ-like peptides must be produced from APLP's (FIG. 8). Thus cleavage of APP and APLP's produces very similar secreted large extracellular proteins (APPₛ, APLP1ₛ, and APLP2ₛ) and soluble intracellular fragments (AICDs) but distinct small secreted peptides that are composed of non-homologous sequences.

Another aspect of the present invention is that because APP and APLP cleavage is physiologically important, inhibition of BACE 1 in brain can interfere with APP and APLP processing, and thereby inhibit their common functions. As the list of substrates of γ-secretase grows, the therapeutic strategy for Alzheimer's disease has shifted from inhibiting γ-secretase to modulating β- and α-secretases. BACE 1 inhibitors have been suggested as a promising cure for Alzheimer's disease. The present disclosure demonstrates, however, that inhibition of BACE 1 can interfere with the functions of APP and APLP's, thus, drugs that target BACE 1 generically can produce unknown side effects. It would be useful, therefore, to identify pharmaceutical agents that are able to modulate the cleavage of APP but to have no effect on cleavage of APLP's.

The abundance of the proteolytic fragments that are produced by APP and APLP cleavage will depend on the regulation of cleavage. If cleavage by β-secretase is upregulated or cleavage of α-secretase downregulated, Aβ production will increase. Thus, identification of the signals and mechanisms involved in regulating the function of APP and APLP is of prime importance since one possible mechanism by which one could interfere with the pathogenesis of Alzheimer's disease would be to alter these signals and mechanisms.

In accordance with these findings, one embodiment of the present invention provides compositions and methods of screening and identifying an agent or agents that modulate the cleavage of APP. Such agents would be prime candidate therapeutics for the treatment of Alzheimer's disease, including naturally occurring or synthetic proteins, peptides, non-peptide small molecules, and any other source of therapeutic candidate agents. Agents identified as affecting APP cleavage may be subsequently tested for biological activity and used as therapeutics or as models for rational drug design.

One embodiment of the present invention provides compositions which are useful in the screening and identifying of an agent or agents that modulate the cleavage of APP. One composition of the present invention comprises a polypeptide substrate for cleavage by β-secretase, wherein the polypeptide substrate includes a transmembrane region and an exogenous APP β-secretase cleavage site inserted near the transmembrane region. In certain embodiments of the present invention, the exogenous β-secretase cleavage site comprises the amino acid sequence EVKMDAE. Alternative embodiments of the present invention also include nucleic acid sequences encoding the polypeptide substrate.

Another embodiment of the compositions provided by the present invention comprises a polypeptide substrate including a transmembrane region and an exogenous APLP1 or APLP2 β-secretase cleavage site inserted into the polypeptide near the transmembrane region. In certain embodiments, the insertion includes the exogenous APLP1 β-secretase cleavage site that is included in the APLP1 linker region sequence DELAPAGTGVSRE and the analogous APLP2 β-secretase cleavage site. Alternative embodiments of the present invention also include nucleic acid sequences encoding the polypeptide substrate.

One embodiment of the present invention provides a method of screening and identifying agents that modulate the cleavage of APP by β-secretase comprising: providing a chimeric molecule, wherein the chimeric molecule includes a transmembrane domain, a β-secretase cleavage site, and an extracellular domain; contacting the chimeric molecule with a β-secretase in the presence or absence of potential cleavage modulating agents; and identifying occurrences of cleavage of the chimeric molecule, wherein a difference in cleavage in the presence of the agent relative to cleavage in the absence of agent is indicative of a cleavage modulating agent. In some embodiments of this method, the β-secretase cleavage site comprises the amino acid sequence EVKMDAE, while in alternate embodiments the β-secretase cleavage site would include an amino acid sequence which corresponds to the amino acid sequences of APLP1 or APLP2 that are responsible for mediating β-secretase cleavage. In certain embodiments, the β-secretase cleavage site will comprise the amino acid sequence DELAPAGTGVSRE.

In certain embodiments, the chimeric molecule comprises the BACE-1 cleavage site and transmembrane domain from APP or the transmembrane domain from any naturally occurring or synthetic integral membrane protein.

In certain embodiments of the present invention, the chimeric molecule is provided by being expressed on the surface of a cell. Cells expressing the chimeric molecule can be stably or transiently transfected with a vector construct encoding the chimeric molecule using methods well known to those of ordinary skill in the art. Suitable vectors for use in such expression as well as methods of creating the construct vector encoding the chimeric molecule are also well known in the art. The cells expressing the chimeric molecule may be any type of cell, preferably mammalian cells, and most preferably human cells. Preferred cells include but are not limited to HEK293 cells, HeLa cells and other mammalian cells that express endogenous BACE 1. In certain embodiments the cell will express endogenous β-secretase, while in more preferred embodiments the cell will endogenously express the β-secretase BACE 1.

In certain embodiments of the present invention, the extracellular domain of the chimeric molecule will serve as a marker for cleavage by β-secretase. In other words, certain features of the extracellular domain will facilitate its identification when it is no longer attached to or retained on the cell allowing it to serve as an indicator of cleavage at the β-secretase cleavage site. The extracellular domain may function as such an identification marker in a wide variety of manners well known to those of ordinary skill in the art. For example, cleavage of the β-secretase site may solubilize the extracellular domain releasing it into the suspension media of the cell culture. The solubilized extracellular domain can then be extracted by removing the cells from the suspension. Once the cells are separated from the suspension media, the presence or absence of solubilized extracellular domains in the media can be analyzed in a variety of quantitative and/or qualitative manners, such as enzyme linked immunosorbant assays (“ELISAs”), flow cytometry, video microscopy, or any suitable high throughput assay known in the art.

In certain embodiments of the present invention, immunostaining, immunoabsorption or immunoblotting assays can be utilized to identify and/or measure solubilized extracellular domain. In certain other embodiments, the extracellular domain of the chimeric molecule comprises a directly or indirectly identifiable marker. For example, in conjunction with such embodiments, tag sequences which are commonly used as epitopes for quantitative immunoassays can be included within the extracellular domain, such as a poly-His region or other antigenic identification peptides. In other embodiments, the extracellular domain comprises a molecule with fluorescent attributes, such as for example green fluorescent protein or luciferase, or comprises a molecule with enzymatic activity that may used to cleave a substrate which produces a colorimetric cleavage product for marker purposes.

An alternate embodiment of the present invention takes advantage of β-secretase cleavage of APP being a rate-limiting step in the cytoplasmic translocation of AICD. Once APP has been cleaved by β-secretase, γ-secretase can cleave APP in the transmembrane region allowing the AICD to undergo translocation to the nucleus. Therefore, assays similar to those disclosed in U.S. 2003/0134323, Sudhof et al., are amenable to use within the present invention. Thus, this alternative embodiment of the present invention provides a method of screening and identifying agents that modulate the cleavage of APP by β-secretase comprising: providing a chimeric molecule, wherein the chimeric molecule includes a transmembrane domain with a γ-secretase cleavage site, a β-secretase cleavage site, and an APP C-terminal cytoplasmic tail modified to allow detection of nuclear localization; contacting the chimeric molecule with a secretase in the presence or absence of potential modulating agents; contacting the chimeric molecule with a γ-secretase; and identifying occurrences of cleavage by measuring nuclear localization of the C-terminal cytoplasmic tail.

Any region of the C-terminal cytoplasmic tail of APP can be modified to allow detection of nuclear localization. In certain embodiments, the cytoplasmic tail of APP can be modified to comprise a DNA binding domain and an activation domain of the same or different heterologous transcription factors. In such an embodiment, nuclear localization would be determined by measuring the activation of transcription of an indicator gene that is under the transcriptional control of a binding site for the DNA binding domain. Such transcription factors and their component DNA-binding and activation domains are well known to those of ordinary skill in the art.

In a certain embodiments, the heterologous DNA-binding domain used can be the yeast transcription factor Ga14, or the bacterial LexA DNA binding domain, which are well known in the art. The cytoplasmic tail modifications may also comprise the transcriptional activation domain of Ga14, and/or another activator such as the viral VP16 activator. In alternative embodiments, a DNA-binding domain without an activation domain is included within the cytoplasmic tail and the mammalian nuclear protein, Fe65, is added to facilitate transcriptional activation. The term Fe65 as used in this disclosure encompasses modifications such as insertions, deletions and substitutions; provided, however, that the functional ability of Fe65 to facilitate transcriptional activation is maintained. Accordingly, the modification of the cytoplasmic tail can consist of a heterologous DNA-binding domain, or consist of a DNA-binding domain and a transcriptional activation domain from the same or different sources.

In these embodiments of the present invention, an indicator gene can be operably linked to a binding site for the DNA-binding protein to facilitate the measuring of nuclear translocation. Indicator genes can be provided in the form of a Ga14 or LexA dependent reporter plasmid containing a marker gene such as luciferase or chloramphenicol acetyl transferase under the control of a Ga14 or LexA regulatory element, respectively, such as an upstream activating sequence. Nuclear translocation of the cytoplasmic tail of APP can result in translocation of the transcription factor as well, causing an activation of transcription of the marker gene. Therefore, detection of the marker gene product provides an assay for the direct detection of nuclear localization of the cytoplasmic tail of APP, and an indirect measure of β-secretase cleavage of APP.

In another embodiment of this method, cells can be co-transfected with plasmids containing nucleic acids encoding APP, Fe65, and modified Tip60 engineered to contain the DNA binding domain of a transcriptional activator, such as Ga14. These cells can, therefore, be co-transfected with the appropriate reporter plasmid as described above to provide an direct measure of nuclear localization of the AICD and an indirect measure of APP cleavage by β-secretase.

Another embodiment of the present invention provides methods of identifying an agent or agents that differentially modulate the cleavage of APP in comparison to the cleavage of APLPs. One such embodiment provides a method of screening and identifying agents that modulate the cleavage of APP by β-secretase comprising: providing an APP chimeric molecule, wherein the APP chimeric molecule includes a transmembrane domain, an APP β-secretase cleavage site, and an extracellular domain; providing an APLP chimeric molecule, wherein the APLP chimeric molecule includes a transmembrane domain, an APLP β-secretase cleavage site and an extracellular domain; contacting both chimeric molecules with a β-secretase in the presence or absence of potential modulating agents; and differentially identifying occurrences of cleavage of the chimeric molecules. In these embodiments, the APP β-secretase cleavage site comprises the amino acid sequence EVKMDAE, while the APLP β-secretase cleavage site would include an amino acid sequence which corresponds to the amino acid sequences of APLP1 or APLP2 that are responsible for mediating β-secretase cleavage. In certain embodiments, the β-secretase cleavage site will comprise the amino acid sequence DELAPAGTGVSRE.

In certain embodiments, the APP chimeric molecule comprises the APP β-secretase cleavage site and the APP transmembrane domain or the transmembrane domain from any naturally occurring or synthetic integral membrane protein. Likewise, the APLP chimeric molecule comprises the APLP β-secretase cleavage site and the APLP transmembrane domain or the transmembrane domain from any naturally occurring or synthetic integral membrane protein.

In certain embodiments of the present invention, the chimeric molecules will be provided by being simultaneously expressed on the surface of a cell. Cells containing chimeric molecules can be stably or transiently transfected with vector constructs encoding the chimeric molecules using methods well known to those of ordinary skill in the art. Suitable vectors for use in such expression as well as methods of creating the construct vectors encoding the chimeric molecules are also well known in the art. Cells expressing the chimeric molecules may be any type of cell, preferably mammalian cells, and most preferably human cells. Preferred cells include but are not limited to HEK293 cells or HeLa cells, however any cell in which BACE 1 can be expressed is useful in the practice of the present disclosure. In certain embodiments the cell will express endogenous β-secretase, while in more preferred embodiments the cell will endogenously express the β-secretase BACE 1.

In certain embodiments of the present invention, the extracellular domain of the chimeric molecules will serve as a marker for cleavage by β-secretase. In other words, certain features of the extracellular domain will facilitate its identification when it is no longer attached or to or retained on the cell allowing it to serve as an indicator of cleavage at the β-secretase cleavage site. In certain embodiments, the extracellular domain used for the APP chimeric molecule will be distinguishable from the extracellular domain used for the APLP chimeric molecule. The extracellular domains can function as such an identification marker in a wide variety of manners well known to those of ordinary skill in the art. For example, cleavage of the β-secretase site can solubilize the extracellular domains releasing it into the suspension media of the cell culture. The solubilized extracellular domains can then be extracted by removing the cells from the suspension. Once the cells are separated from the suspension media, the presence or absence of solubilized extracellular domains in the media can be analyzed in a variety of quantitative or qualitative manners, such as for example ELISAs, flow cytometry, video microscopy or other high throughput assays known in the art.

In certain embodiments of the present invention, immunostaining, immunoabsorption or immunoblotting assays can be utilized to differentially measure and/or identify solubilized extracellular domains. In certain other embodiments, the extracellular domain itself of the chimeric molecule comprises a directly or indirectly identifiable marker. In conjunction with such embodiments, various different tag sequences which are commonly used as epitopes for quantitative immunoassays can be included within the extracellular domains, such as, for example, a poly-His region or other antigenic identification peptides. In alternate embodiments, an extracellular domain comprises a molecule with fluorescent attributes, such green fluorescent protein or luciferase, or comprises a molecule with enzymatic activity that can used to cleave a substrate which produces a colorimetric cleavage product for marker purposes. In preferred embodiments, the extracellular domain of one of the specific chimeric molecules, for example the APP chimeric molecule, would comprise one colorimetric readout such as green, while the other chimeric molecule, in this example APLP, would comprises a different colorimetric readout, such as red.

An alternate embodiment of the present invention takes advantage of β-secretase cleavage of APP being a rate-limiting step in the cytoplasmic translocation of the AICD. Once APP has been cleaved by β-secretase, γ-secretase is can cleave APP in the transmembrane region allowing the AICD to undergo translocation to the nucleus. Therefore, assays similar to those disclosed in U.S. 2003/0134323, Sudhof et al., are amenable to use within the present invention. Thus, an alternative embodiment of the present invention provides a method of screening and identifying agents that differentially modulate the cleavage of APP by β-secretase as compared to APLP comprising: providing an APP chimeric molecule, wherein the APP chimeric molecule includes a transmembrane domain including a γ-secretase cleavage site, an APP β-secretase cleavage site, and an APP C-terminal cytoplasmic tail modified to allow detection of nuclear localization; providing an APLP chimeric molecule, wherein the APLP chimeric molecule includes a transmembrane domain having a γ-secretase cleavage site, an APLP β-secretase cleavage site, and an APP C-terminal cytoplasmic tail modified to allow detection of nuclear localization; contacting the chimeric molecules with a β-secretase in the presence or absence of potential modulating agents; contacting the chimeric molecules with a γ-secretase; and identifying occurrences of cleavage by differentially measuring nuclear localization of the C-terminal cytoplasmic tail from the APP chimeric molecule and the APLP chimeric molecule, wherein a difference in cleavage in the presence of the agent relative to cleavage in the absence of the agent is indicative of a cleavage modulating agent. In certain alternate embodiments, the APLP chimeric molecule comprises an APLP C-terminal cytoplasmic tail instead of an APP C-terminal cytoplasmic tail.

Any region of the C-terminal cytoplasmic tail of APP can be modified to allow detection of nuclear localization. In certain embodiments, the cytoplasmic tail of APP can be modified to comprise a DNA binding domain and an activation domain of the same or different heterologous transcription factors. In such an embodiment, nuclear localization would be determined by measuring the activation of transcription of an indicator gene that is under the transcriptional control of a binding site for the DNA binding domain. Such transcription factors and their component DNA-binding and activation domains are well known to those of ordinary skill in the art. The use of different DNA binding domains and activation domains on the APP and APLP chimeric molecules will enable differential measurements of translocation of the cytoplasmic tail for the two types of chimeric molecules.

In certain embodiments, the heterologous DNA-binding domain may be the yeast transcription factor Ga14, or the bacterial LexA DNA binding domain, which are well known in the art. The cytoplasmic tail modifications can also comprise the transcriptional activation domain of Ga14, and/or another activator such as the viral VP16 activator. In alternative embodiments, a DNA-binding domain without an activation domain is included within the cytoplasmic tail and the mammalian nuclear protein, Fe65, is added to facilitate transcriptional activation. The term Fe65 as used in this disclosure encompasses modifications such as insertions, deletions and substitutions; provided however, that the functional ability of Fe65 to facilitate transcriptional activation is maintained. Accordingly, the modification of the cytoplasmic tail can consist of a heterologous DNA-binding domain, or consist of a DNA-binding domain and a transcriptional activation domain from the same or different sources.

In these embodiments of the present invention, an indicator gene can be operably linked to a binding site for the DNA-binding protein to facilitate the measuring of nuclear translocation. Indicator genes can be provided in the form of a Ga14 or LexA dependent reporter plasmid containing a marker gene such as luciferase or chloramphenicol acetyl transferase under the control of a Ga14 or LexA regulatory element, respectively, such as an upstream activating sequence. Again the use of different DNA-binding domains in conjunction with different respective marker genes will facilitate the differential measuring of translocation between the APP and APLP chimeric molecule cytoplasmic tails. Nuclear translocation of the cytoplasmic tail can result in translocation of the transcription factor as well, causing an activation of transcription of the marker gene. Therefore, detection of a marker gene product provides an assay for the direct detection of nuclear localization of the cytoplasmic tail of a particular chimeric molecule, and an indirect measure of β-secretase cleavage.

Agents identified through the use of the methods of the present invention may be useful in the development of treatments for patients having Alzheimer's disease. Furthermore, the methods of the present invention are amenable to use in high throughput formats in order facilitate the search for such agents. Agents identified through the methods of the present invention can be subsequently tested for biological activity and used as therapeutics or therapeutic models.

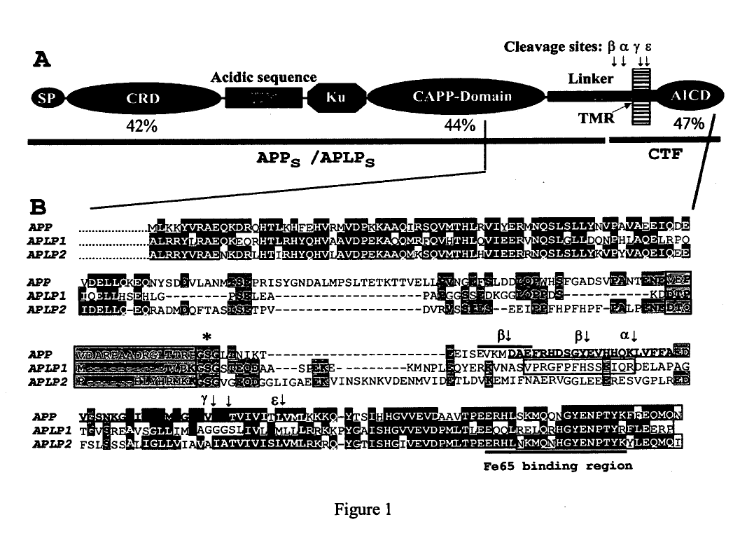

A further aspect of the present disclosure is a, biological activity associated with the central APP domain (CAD) as shown in FIG. 1A. The present inventors have discovered that F-spondin is an extracellular ligand that binds to this domain of APP and the APP like proteins APLP1 and APLP2. F-spondin is a secreted multi-domain protein that promotes neural cell adhesion and neurite extension. F-spondin is composed of an N-terminal 200 residue region that is homologous to reelin, a central “Spondin” domain and six C-terminal thrombospondin type 1 repeats. F-spondin is expressed at high levels in the floor plate of the developing spinal cord, but is also ubiquitously present in embryonic and adult tissues.

F-spondin is expressed at highest levels in developing neurons, but axotomy of adult sciatic nerve causes massive upregulation of F-spondin. Recombinant F-spondin promotes neural cell adhesion and neurite extension, suggesting that it may function to stimulate axonal extension and repair. F-spondin binds to the cell surface of neurons, but no neuronal receptor has been identified. Recombinant F-spondin also stimulates proliferation of vascular smooth muscle cells, suggesting that consistent with its ubiquitous expression, F-spondin also acts on non-neuronal cells. Thus, F-spondin likely mediates cellular responses in brain and periphery by binding to specific cell-surface receptors. It is contemplated therefore, that APP may serve as an F-spondin receptor. During investigation of this idea, the present inventors discovered that F-spondin, and more particularly the reelin domain or the reelin and spondin domains are sufficient for binding to the CAD domain APP. It was further discovered that this binding inhibits the cleavage at the β-secretase cleavage sites.

The following nonlimiting examples serve to further illustrate the present invention.

### EXAMPLES

Experimental Procedures for Examples:

Plasmids.

Most plasmids used for the transactivation assays, such as pG5E1B-luc (Ga14 reporter plasmid), pCMV-LacZ (β-galactosidase control plasmid), pM-Tip60 (expression of Ga14-Tip60 fusion protein), pCMV-Fe65 and pCMV-APP (APP), were reported previously (Cao, X., and Südhof, T. C. (2001) Science 293, 115-120). pcDNA3.1-APLP2 encoding APLP2 was constructed by inserting a 3.0 kb DNA fragment from pOTB7-hAPLP2 (Image clone ID:2820109) into the BamHI and XbaI sites of pcDNA3.1.pCMV-Sport6-APLP1 encoding APLP1 was acquired from ATCC (Image ID:3865417). pCMV-SynCam and pCMV-Nrx1 β were described previously (Biederer, T., et al. (2002) Science 297, 1525-1531; Ushkaiyov, Y. A., et al. (1994) J. Biol. Chem. 269-11987-11992). pCMV-Nrx1β-BS encoding neurexin 1β with 7 residues from APP was generated by inserting synthetic oligonucleotides (sequences: QL0311=GGC CGA GAA GTG AAG ATG GAT GCA GAA AGC; QL0312=GGC CGC TTT CTG CAT CCA GGT TCA CTT CTC) into the Not 1 site of pCMV-Nrx1β. pCMV-NrxAPP was constructed by ligating a 0.6 kb fragment encoding residues 494-695 of APP₆₉₅ into the Not 1 site of pCMV-Nrx1β. pcDNA3.1-ADAM 9 encodes ADAM 9 and pcDNA3.1-Myc-BACE encodes BACE.

Antibodies.

APP, APLP1, and APLP2 antibodies were raised in rabbits to synthetic peptides containing their C-terminal sequences (APPc=U955: GYENPTYKFFEQMQN; APLP1c=U2976: YENPTYRFLEERP; APLP2c=U2977: NKMQNHGYENPTYKYLEQMQI) and to an interior peptide from APLP1 (APLPIx=U4787; residues 554-567: VPRGEPFHSSEIQR). Monoclonal antibodies to the extracellular domain of APP (5A3 and 1G7 [38]) were a kind gift of E. H. Koo (La Jolla, Calif.). All other antibodies were described previously (Cao, X., and Südhof, T. C. (2001) Science 293, 115-120.; Biederer, T., et al. (2002) Science 297, 1525-1531.; Ushkaiyov, Y. A., et al. (1992) Science 257, 50-56.).

Transfection and transactivation assays.

HEK293, HeLa and COS7 cells were transfected at 50 to 80% confluency in 6-well plates using Fugene6 reagent (Roche). For Ga14-Tip60 transactivation assays, HEK293 cells were co-transfected with five plasmids: (1) pG5E1B-luc=0.15 μg; (2) pCMV-LacZ=0.10 μg; (3) pM-Tip60=0.5 μg; (4) pCMV-FE65=0.5 μg; and (5) 0.05-1.0 μg pCMV-APP, 0.025-0.5 μg pCMV-Sport6-APLP1, or 0.05-1.0 μg pcDNA3.1-APLP2 as indicated. For the Fe65 titration experiments, HEK293 cells were cotransfected with five plasmids: (1) pG5E1B-luc=0.15 μg; (2) pCMV-LacZ=0.10 μg; (3) pM-Tip60=0.5 μg; (4) 0.5 μg of APP, 0.25 μg of APLP1, or 0.5 μg of APLP2; and (5) 0-2.0 μg pCMV-Fe65. Cells were harvested 2 days after transfection in 0.25 ml/well of reporter lysis buffer (Promega), and their Luciferase and β-galctosidase activities were determined with the Promega luciferase assay kit using a chemiluminescence reader from Berthold, and with the O-nitrophenyl-D-galacto-pyranoside assay for β-galactosidase, respectively. The luciferase activity was standardized by the β-galactosidase activity to control for transfection efficiency and further normalized with the transactivation level with cells expressing no APPs or Fe65. Values shown are averages of transactivation assays performed in triplates. For immunoblotting analyses, HEK293 were transfected with 1 μg expression plasmids encoding APP, APLP1, APLP2, SynCam, or neurexin 1β, with or without 0.25 μg of BACE 1 expression vector. For APLP1 cleavage experiments, 1 μg of APLP1 or APP was cotransfected with BACE (0.25 μg and 0.5 μg) or ADAM 9 (0.5 μg and 1.0 μg) in HEK293, HeLa and COS cells. The cell extracts or media were harvested 24 hours after the transfection. Where indicated, γ-secretase inhibitor IX (DAPT; Calbiochem) was added (2 μM final concentration) 6 hrs before harvesting the cells.

SDS-PAGE and Immunoblotting.

SDS-PAGE and Immunoblotting experiments were performed using standard procedures well know to one of ordinary skill in the are with either regular SDS gels or tricine gels. Lanes were loaded with ˜20 μg of protein from whole cell extracts harvested in sample buffer, and analysed by immunoblotting after SDS-PAGE using antibody dilutions of 1:1,000-2,000 for primary and 1:10,000 HRP conjugated goat-anti-rabbit secondary antibodies (ICN). For immunoblotting analysis of secreted proteins, supernatants from transfected cells (30 μl with approximately 5 μg protein) were loaded per lane.

### Example 1

Coordinate Transcriptional Activity of APP, APLP1, and APLP2.

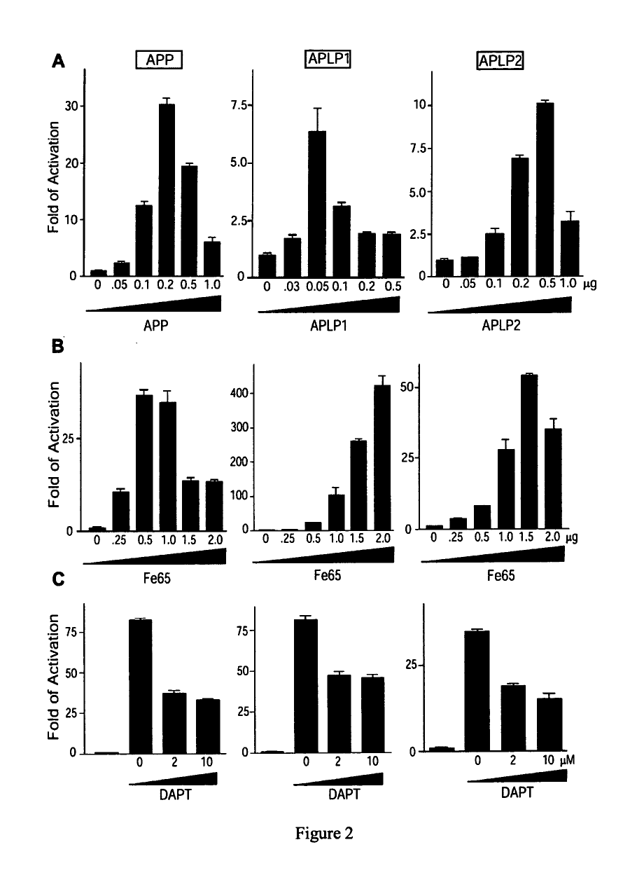

Ga14-Tip60, although part of a nuclear protein complex that binds DNA, is unable to mediate Ga14-dependent transcription but requires both Fe65 and APP for transactivation. The Ga14-Tip60 assay is a more stringent test for the transcriptional function of APP than the use of APP-Ga14 fusion proteins because in the Ga14-Tip60 assay, APP is not itself modified. To determine whether APLP1 and APLP2—which were previously shown as Ga14-fusion proteins to transactivate transcription in an Fe65-dependent manner—can substitute for APP in the Ga14-Tip60 assay and to test whether Tip60 is also a potential target for APLPs, HEK293 cells were co-transfected with increasing amounts of expression plasmids encoding APP, APLP1, or APLP2 and with a constant amount of plasmids expressing Ga14-Tip60 and Fe65. Ga14-dependent transactivation was then measured using a co-transfected reporter plasmid, and all transactivation data for β-galactosidase activity produced was corrected by using a co-transfected control expression vector to account for differences in transfection efficiency (FIG. 2).

FIG. 2A shows the results of transactivation of transcription mediated by Ga14-Tip60 in HEK293 cells that were co-transfected with a constant amount of Fe65 plasmid (0.5 μg) and increasing amounts of APP, APLP1, or APLP2 plasmids as indicated. For all three proteins (APP, APLP1, and APLP2), a bell-shaped dose-response curve of transactivation that depended on APP or an APLP was observed (FIG. 2A). A similar analysis with a constant amount of APP or APLP1 and APLP2, but increasing amounts of Fe65 (FIG. 2B; APP (0.5 μg), APLP1 (0.25 μg), and APLP2 plasmids (0.5 μg)) was also performed. Again, a dose-dependent, highly specific transactivation of Ga14-dependent transcription was detected. The numbers below the bars indicate the amount of the variable plasmid transfected, and all cells were additionally transfected with Ga14-Tip60 expression vector (0.5 μg), the Ga14-reporter plasmid pG5E1B-luc (0.15 μg), and a control plasmid to correct for transfection efficiency (0.10 μg of pCMV-LacZ). All data were corrected for transfection efficiency and normalized to the amount of transactivation observed in the absence of APP or APLP's. Data shown are means+/−S.E.M.'s of triplicates from a representative experiment. The absolute amount of transactivation varied greatly depending on the precise combination of plasmids co-transfected. The highest levels of transactivation (400 fold increase) were reached when maximal amounts of Fe65 were co-transfected with APLP1 (FIG. 2B). To test whether transactivationmediated by APP, APLP1 or APLP2 in these assays involves γ-secretase cleavage, the effect of the γ-secretase inhibitor, DAPT, on transactivation was examined (FIG. 2C). Two relatively low concentrations of DAPT (2 and 10 μM) were used. Significant inhibition of transactivation by DAPT was observed for all three proteins (APP, APLP1 and APLP2). Inhibition was incomplete, possibly because the doses of DAPT do not fully inhibit γ-secretase or because a DAPT-independent pretoelytic pathway contributes to transactivation. Together these results document that APLP1 and APLP2 can efficiently substitute for APP in Tip60-dependent transactivation, suggesting that consistent with a common transcriptional function for APP and APLP's, Tip60 is a common nuclear target.

### Example 2

BACE 1 Cleavage of APLP's.

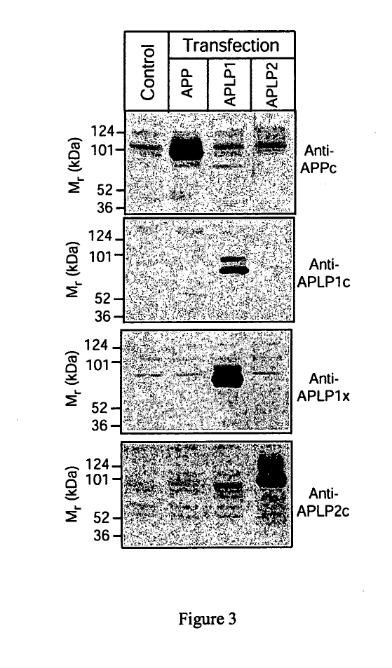

APP and APLP's presumably can only mediate nuclear transactivation if they are cleaved and their AICD's are released into the cytoplasm. APLP's are known to be cut by extracellular proteases and a presenilin-dependent γ-secretase similar to APP. In non-neuronal cells, several proteases that belong to the ADAM family of metalloproteases are thought to cleave APP as α-secretases, whereas in neurons, APP is thought to be primarily cleaved primarily by the β-secretase enzyme BACE 1 whose activity is responsible for production of the pathogenic Aβ peptides. Although APLP's are cleaved extracellularly in non-neuronal and neuronal cells, it is not known whether the same enzymes cleave APP and APLP's. This question is particularly important for BACE 1 for which no substrate other than APP is currently known. In preparation to examine APLP cleavage, antibodies to the cytoplasmic C-terminal sequences of APP, APLP1, and APLP2 were generated. In addition, an antibody to a peptide from the linker region of APLP1 that connects the TMR to the central domain (boxed sequence in FIG. 1B) was produced. The specificity of these antibodies was then tested by immunoblotting of COS cells that had been transfected with APP, APLP1, or APLP2 expression vectors (FIG. 3). Briefly, proteins from HEK293 control cells or cells transfected with APP, APLP1 or APLP2 were analyzed by immunoblotting with various antibodies to the C-terminal sequences of APP (APPc), APLP1 (APLP1c), or APLP2 (APLP2c) or to an internal APLP1 sequence located 13 residues N-terminal to the TMR (APLP 1×). All antibodies were raised against synthetic peptides; epitope sequences are boxed in the sequence alignment shown in FIG. 1B. Numbers on the left indicate the position of molecular weight markers. It was found that the antibodies reacted specifically with their cognate proteins except for a small degree of crossreactivity of the APLP2 antibody with APP and APLP1.

In the next set of experiments these antibodies were employed to probe for APP, APLP1, and APLP2 cleavage in cells that either expressed these proteins alone or in combination with BACE 1 (FIG. 4). As negative controls, two other neuronal cell-surface proteins, neurexin 10 (Nrx1β) and SynCAM (36,37,39) were examined. For each of the five proteins studied, six conditions were investigated: HEK293 control cells (lane 1), transfected cells only expressing BACE 1 (lane 2), or cells transfected either with APP, APLP1, APLP2, neurexin 1β (Nrx1β), or SynCAM alone (lanes 3 and 5) or together with BACE 1 (lanes 4 and 6) were incubated in the absence (lanes 1-4) or presence of the γ-secretase inhibitor DAPT (2 mM; lanes 5-6). Transfected proteins (indicated on the right) were examined by immunoblotting (20 μg cell protein/lane) using regular SDS-PAGE (top panel for each protein) to monitor the full-length proteins (FL), or by tricine gel electrophoresis to detect their CTFs (bottom panel for each protein). All transfected proteins are type I transmembrane proteins, and all immunoblotting was earned out with antibodies to the cytoplasmic C-termini of the proteins. Non-transfected cells and cells transfected only with BACE 1 (lanes 1-2) were used as controls to ensure that the various antibodies did not recognize an endogenous or BACE 1-dependent signal. Asterisks in lane 4 for APP, APLP1, and APLP2 indicate BACE 1-dependent CTFs. Numbers on the left display positions of molecular weight markers. Data shown are from a representative experiment independently repeated multiple times. The DAPT treatments were performed to stabilize the CTFs, proteolytic intermediates that are produced by extracellular α-/β-cleavage of a cell-surface protein and are subsequently digested by γ-secretase. The transfected proteins were then analysed by immunoblotting to monitor the full-length (FL) proteins and their CTFs as a function of BACE 1 and DAPT treatment.

Immunoblotting of the control cells showed that transfected BACE 1 by itself did not produce immunoreactivity with any of the antibodies (lanes 1-2, FIG. 4). In transfected test cells incubated without DAPT, CTFs were observed only for APP and the APLP's but not for neurexins 1β or SynCAM (lanes 3-4). Under these conditions, co-transfection of BACE 1 had little effect on full-length APP or ALP's, but altered their CTFs: For APP and APLP2, the size of the CTF was shifted up, whereas for APLP1 a distinctive CTF was first induced by BACE 1 expression (asterisks in lane 4, FIG. 4). When DAPT was added, the CTFs for APP, APLP1, and APLP2 became very abundant independent of BACE 1 expression, making it difficult to detect an effect of BACE 1 (lanes 5-6). CTFs were also observed for neurexin 1β upon DAPT treatment, most likely an artifact of the expressed protein because BACE 1 had no effect on the production and abundance of CTFs for either neurexin 1β or SynCAM. Together these data suggest that in the absence of BACE1, APP and APLP's are efficiently cleaved in transfected cells by secretases, likely endogenous α-secretases, but that BACE 1 expression alters cleavage of all three proteins, indicating that all three proteins are BACE 1 substrates.

To confirm that BACE 1 cleaves APLP's, the effect of BACE 1 co-transfection on Ga14-Tip60 dependent transactivation by APLP1 and Fe65 was examined in an assay similar to the one described in FIG. 2. Co-transfecting a small amount of APLP1 with Fe65 and Ga14-Tip60 resulted in a small degree of transactivation (5 fold). However, adding increasing amounts of BACE 1 expression plasmid to the co-transfection mix caused a proportional increase in transactivation (FIG. 5), indicating that BACE 1 cleavage of APLP1 contributes to Ga14-Tip60 dependent transcription. In FIG. 5, HEK293 cells were co-transfected with constant amounts of Ga14-Tip60, Ga14-reporter plasmid, and γ-galactosidase (to control for transfection efficiency), with or without APLP1 as indicated, and with increasing amounts of BACE 1 expression plasmid. Data shown represent fold transactivation over Ga14-Tip60 alone corrected for transfection efficiency.

### Example 3

BACE 1 Cleavage Specificity

The cleavage of APLP1 and APLP2 by BACE was unexpected considering the lack of sequence homology in the APP cleavage site for BACE 1 (FIG. 1B). Although the co-transfection/cleavage assay for BACE 1 cleavage has been used previously to confirm BACE 1 enzyme activity, the specificity of BACE 1-mediated cleavage under these conditions has not been investigated, and the substrate specificity of BACE 1 has been studied primarily with short synthetic peptides. The data in FIG. 4 demonstrate that BACE 1 does not randomly cleave overexpressed cell-surface proteins since neurexin 1β and SynCAM were not digested. However, the data raises the question of what determines the cleavage specificity of BACE 1: is cleavage a property of a specific sequence in APP, possibly in combination with the proximity of that sequence to the membrane, or are the extra- or intracellular domains of APP involved in directed BACE 1 cleavage? To address these questions, a hybrid neurexin 1β/APP protein in which the extracellular sequences of neurexin 1β are fused to the C-terminal sequences of APP at a position just N-terminal to the normal BACE 1 cleavage site was constructed. FIG. 6A, shows diagrams of the structures of APP, the neurexin 1′/APP hybrid protein (Nrx1β/APP), and the modified neurexin 1β (Nrx1β*) containing a seven residue insertion (EVKMDAE) from APP into neurexin 1β just N-terminal to the TMR. The β-secretase cleavage site containing the Swedish mutation (EVNLDAES) that is preferentially cleaved by BACE 1 was also inserted. In the neurexin 1β/APP hybrid protein, the nearly complete extracellular sequence of neurexin 1β (residues 1-375) is fused to APP such that only a short sequence of the extracellular linker from APP ( . . . residues) followed by the TMR and AICD from APP are included (residues 494-695 of APP₆₉₅). Comparison of the cleavage of APP and of the neurexin/APP hybrid protein in transfected cells revealed that they were almost identically processed, with a similar production of CTFs that were significantly shifted to larger sizes by co-expression of BACE 1. FIG. 6B shows a comparison of the effect of BACE 1 and DAPT on CTFs produced in HEK293 control cells (lanes 1-2) or transfected cells expressing APP (lanes 3-6) or the neurexin 1β/APP hybrid (lanes 7-10). Cells transfected and treated as indicated were examined by immunoblotting after standard gel electrophesis (top panel) or tricine gel electrophoresis (bottom panel) with antibodies to the C-terminus of APP. For both APP and the APP/neurexin hybrid, DAPT induced a massive accumulation of CTFs consistent with normal digestion of the CTFs by γ-secretase (lanes 4, 6, 8, and 10). Thus the extracellular domains of APP are dispensible for BACE 1 cleavage.

The ability of intracellular AICD or the TMR of APP to direct BACE 1-was tested by inserting a seven residue sequence from APP that encompasses the normal BACE 1 cleavage site (sequence: EVKMDAE) into full-length neurexin 1β. The insertion was placed just outside of the TMR, corresponding to the normal position of this sequence in APP, but no other APP-related sequences were present in this neurexin 1β derivative (referred to as Nrx1β*). Analysis of co-transfected cells revealed that insertion of the short 7 residue sequence was sufficient to convert neurexin 1β into a BACE 1 substrate (FIG. 6C; same as B., except that the effect of BACE 1 on the cleavage of neurexin 1β and neurexin 1β* was examined): Now CTFs are observed as a function of BACE 1 in the absence of DAPT (compare lanes 7 and 9), and the amount of CTFs is greatly increased by co-expression of BACE 1 in the presence of DAPT (lanes 8 and 10). BACE 1 cleavage of Nrx1β* was so efficient that the amount of full-length Nrx1β* protein dropped precipitously in cells co-transfected with BACE 1 (FIG. 6C). These data demonstrate that even in the context of a resident membrane protein, a very short substrate sequence is sufficient to confer BACE 1 cleavage onto a protein.

### Example 4

Position of BACE 1 Cleavage of APLP1

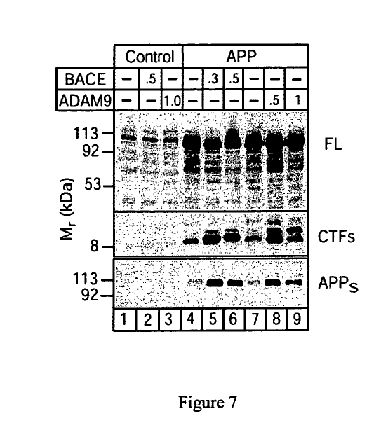

BACE 1 cleavage appears to be relatively non-specific because APLP1 and APLP2 are also cleaved by BACE 1 at a site that exhibits no sequence similarity with the APP cleavage site for BACE 1. The possibility that all extracellular proteases that cleave APP also digest APLP's was addressed by a more detailed study of the cleavage of APLP1 (chosen because it is less similar to APP than APLP2) and by comparing the activity of BACE 1 with that of ADAM 9, a disintegrin metalloprotease that is thought to be one of several α-secretase enzymes. To validate the activity of BACE 1 and ADAM 9, APP was examined first. Briefly, in FIG. 7, the panels show immunoblots from HEK293 control cells (lane 1), cells transfected with the indicated amount of BACE 1 or ADAM 9 alone (lanes 2 and 3, respectively), or cells transfected either with APP alone (lanes 4 and 7) or together with two concentrations of BACE 1 plasmid (lanes 5-6) or ADAM 9 plasmid (lanes 8-9). Blots show analysis of cellular proteins to detect full-length APP (FL) separated on regular SDS gels (top) and CTFs separated on tricine gels (middle panel). In addition, the media of the cells was collected and analysed for the secreted proteolytic APP-fragment (APPₛ bottom panel). The top two blots were obtained with antibodies to the cytoplasmic C-terminus of APP, and the bottom blot with antibodies to extracellular APP sequences.

Transfection of BACE 1 or ADAM 9 alone into HEK293 cells had no significant effect on the processing of endogenous APP in cells, probably because the majority of the endogenous APP is from non-transfected cells (lanes 1-3, FIG. 7). Without BACE 1 or ADAM 9, a single major CTF was observed from transfected APP at steady-state (lane 4, FIG. 7). Co-transfection of APP with BACE 1 at two concentrations (lanes 5-6) or ADAM 9 at two concentrations (lanes 8-9) increased the abundance of the APP CTF, consistent with cleavage of APP by both transfected BACE 1 and ADAM 9. In parallel, APPₛ—the secreted extracellular fragment of APP that results from α-/β-cleavage—was monitored with antibodies to the extracellular APP sequences in the medium from the transfected cells. A dramatic increase in APPₛ production was observed both after BACE 1 and after ADAM 9 cotransfection (FIG. 7). Together these results demonstrate that in co-transfected cells, both BACE 1 and ADAM 9 cleave APP and increase secretion of APPₛ.

Similar experiments were then performed for APLP1 (FIG. 8), probing the intracellular sequence with the antibody to the C-terminus used above, and the secreted extracellular domain sequences (APLP1ₛ) with a new polyclonal antibody that was raised to a short synthetic peptide derived from the extracellular sequence of APLP1 just outside of the TMR (boxed in FIG. 1B). Briefly, in FIG. 8, the panels show immunoblots from control cells (lane 1), cells transfected with the indicated amount of BACE 1 or ADAM 9 alone (lanes 2 and 3, respectively), or cells transfected either with APLP1 alone (lanes 4 and 7) or together with two concentrations of BACE 1 plasmid (lanes 5-6) or ADAM 9 plasmid (lanes 8-9). Three different cell line were analysed to control for possible cell-type specific effects on cleavage. Blots show analysis of cellular proteins to detect full-length APLP1 (FL) separated on regular SDS gels (top) and CTFs separated on tricine gels (middle panel). In addition, the media of the cells was collected and analysed for the secreted proteolytic APLP1-fragment (APLP1ₛ) in the bottom panel. The top two blots were obtained with antibodies to the cytoplasmic C-terminus of APLP1, and the bottom blot with antibodies to a short extracellular peptide sequence of APLP1 that is located 13 residues N-terminal to the TMR (boxed in FIG. 1B). Numbers on the left indicate positions of molecular weight markers.

Three different cell lines (HEK293, HeLa, and COS cells) were tested to exclude cell-specific artifacts. Indeed, slight differences in the properties of the CTFs produced in the different lines consistent with differences in the presence of various α-secretases were observed. Nevertheless, in all cell lines co-expression of BACE 1 caused a large change in CTFs whereas co-expression of ADAM 9 had no detectable effect (FIG. 8). Furthermore, no APLP1ₛ was detectable in the medium from transfected cells except after BACE 1 expression. Since the antibody to the extracellular APLP1 sequences used in this experiment is to a sequence that is very close to the TMR (FIG. 1B), the possibility that an APLP1ₛ fragment is produced in the absence of BACE 1 in APLP1 transfected cells by a secretase that cleaves N-terminal to the antibody used cannot be ruled out. However, the clear-cut appearance of the APLPₛ fragment after BACE 1 expression demonstrates that BACE 1 cleaves APLP1 in the 13 residues that are located between the epitope of the antibody and the TMR (FIG. 1B).

1. A method of identifying agents that modulate the cleavage of APP by a β-secretase comprising:
(a) providing a chimeric molecule, wherein the chimeric molecule includes a transmembrane region, an APP β-secretase cleavage site, and an extracellular region;
(b) contacting the chimeric molecule with a β-secretase in the presence and absence of at least one potential cleavage modulating agent; and
(c) identifying occurrences of cleavage of the chimeric molecule;
wherein a difference in cleavage in the presence of the agent relative to cleavage in the absence of the agent is indicative of a cleavage modulating agent.

2. The method of claim 1, wherein the β-secretase cleavage site is the amino acid sequence EVKMDAE.

3. The method of claim 1, wherein the β-secretase cleavage site is the amino acid sequence EVNLDAE.

4. The method of claim 1, wherein the chimeric protein is expressed from an expression vector.

5. The method of claim 4, wherein the chimeric protein is expressed in a host cell.

6. The method of claim 5, wherein the host cell expresses an active β-secretase enzyme.

7. The method of claim 6, wherein the host cell expresses an endogenous β-secretase enzyme.

8. The method of claim 6, wherein the host cell comprises an expression vector that expresses β-secretase.

9. The method of claim 1, wherein identifying occurrences of cleavage comprises detection of a cleaved extracellular region.

10. The method of claim 1, wherein the extracellular region includes the central APP domain (CAD).

11. The method of claim 1, wherein the extracellular region binds F-spondin.

12. A method of identifying agents that modulate the cleavage of APP by a β-secretase comprising:
(a) providing a chimeric molecule, wherein the chimeric molecule includes a transmembrane region with a γ-secretase cleavage site, a β-secretase cleavage site, and an APP C-terminal cytoplasmic tail modified to allow detection of nuclear localization;
(b) contacting the chimeric molecule with a β-secretase in the presence and absence of at least one potential modulating agent;
(c) contacting the chimeric molecule with a γ-secretase; and
(d) identifying occurrences of cleavage by measuring nuclear localization of the C-terminal cytoplasmic tail
wherein a difference in cleavage in the presence of the agent relative to cleavage in the absence of the agent is indicative of a cleavage modulating agent.

13. The method of claim 12, wherein the β-secretase cleavage site is the amino acid sequence EVKMDAE.

14. The method of claim 12, wherein the chimeric protein is expressed from an expression vector.

15. The method of claim 14, wherein the protein is expressed in a host cell.

16. The method of claim 15, wherein the host cell expresses an active β-secretase enzyme.

17. The method of claim 15, wherein the host cell expresses an endogenous β-secretase enzyme.

18. The method of claim 15, wherein the host cell comprises an expression vector that expresses β-secretase.

19. A method of identifying agents that specifically modulate the cleavage of APP by β-secretase with respect to cleavage of APLP comprising contacting an APLP with β-secretase in the presence and absence of a modulator of β-secretase cleavage of APP, wherein lack of a significant difference in cleavage of the APLP in the presence and absence of the modulator is indicative of a specific modulator of β-cleavage of APP.

20. The method of claim 19, wherein the APLP is APLP1.

21. The method of claim 19, wherein the APLP is APLP2.

22. A composition comprising a polypeptide substrate for cleavage by β-secretase comprising a transmembrane region and an exogenous APP β-secretase cleavage site inserted into the polypeptide near the transmembrane region.

23. The composition of claim 22, wherein the exogenous β-secreatase cleavage site is inserted from 1 to 100 residues from the transmembrane region.

24. The composition of claim 22, wherein the exogenous β-secreatase cleavage site is inserted from 10 to 90 residues from the transmembrane region.

25. The composition of claim 22, wherein the exogenous β-secreatase cleavage site is inserted from 40 to 50 residues from the transmembrane region.

26. The composition of claim 22, wherein the β-secreatase cleavage site is the amino acid sequence EVKMDAE.

27. An isolated nucleic acid encoding the polypeptide substrate of claim 22.

28. A host cell comprising the nucleic acid of claim 27.

29. The host cell of claim 28, further defined as a mammalian cell.

30. A composition comprising a polypeptide substrate for cleavage by β-secretase comprising a transmembrane region and an exogenous APLP1 β-secretase cleavage site inserted into the polypeptide near the transmembrane region.

31. The composition of claim 30, wherein the exogenous β-secreatase cleavage site is inserted from 1 to 100 residues from the transmembrane region.

32. The composition of claim 30, wherein the exogenous β-secreatase cleavage site is inserted from 10 to 90 residues from the transmembrane region.

33. The composition of claim 30, wherein the exogenous β-secreatase cleavage site is inserted from 40 to 50 residues from the transmembrane region.

34. The composition of claim 30, wherein the β-secreatase cleavage site is the amino acid sequence DELAPAGTGVSRE.

35. An isolated nucleic acid encoding the polypeptide substrate of claim 30.

36. A host cell comprising the nucleic acid of claim 35.

37. The host cell of claim 36, further defined as a mammalian cell.

38. A method of identifying agents that modulate the cleavage of APP like proteins by a secretase comprising:
(a) providing a chimeric molecule, wherein the chimeric molecule includes a transmembrane region, an APLP β-secretase cleavage site, and an extracellular region;
(b) contacting the chimeric molecule with a β-secretase in the presence and absence of at least one potential cleavage modulating agent; and
(c) identifying occurrences of cleavage of the chimeric molecule;
wherein a difference in cleavage in the presence of the agent relative to cleavage in the absence of the agent is indicative of a cleavage modulating agent.

39. The method of claim 38, wherein the APLP β-secretase cleavage site is an APLP1 cleavage site.

40. The method of claim 38, wherein the APLP β-secretase cleavage site is an APLP2 cleavage site.

41. The method of claim 38, wherein the chimeric protein is expressed from an expression vector.

42. The method of claim 41, wherein the protein is expressed in a host cell.

43. The method of claim 42, wherein the host cell expresses an active β-secretase enzyme.

44. The method of claim 43, wherein the host cell expresses an endogenous β-secretase enzyme.

45. The method of claim 43, wherein the host cell comprises an expression vector that expresses β-secretase.

46. The method of claim 38, wherein identifying occurrences of cleavage comprises detection of a cleaved extracellular region.

47. The method of claim 38, wherein the extracellular region includes the central APP domain (CAD).

48. The method of claim 38, wherein the extracellular region binds F-spondin.
